(19) United States
(12) Patent Application Publication
(10) Pub. No.: US 2008/0075661 A1
(43) Pub. Date: Mar. 27, 2008

(54) Compounds, Kits and Methods for Use in Medical Imaging

(75) Inventors: Marc Stefan Robillard, Eindhoven (NL); Holger Gruell, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/576,537

(22) PCT Filed: Oct. 4, 2005
(86) PCT No.: PCT/IB05/53257

§ 371 Date: Apr. 3, 2007

(30) Foreign Application Priority Data
Oct. 7, 2004 (EP) 04104913.1

(51) Int. Cl. A61K 49/00

(52) U.S. Cl. 424/9.1

(57) ABSTRACT

FDG alternatives are provided. They consist of a two component system comprising on the one hand a building block such as glucose linked to an azide, alkyne or phosphine and on the other hand a detectable label linked to an azide, alkyne or phosphine which is the counterpart of the group linked to glucose in a Staudinger ligation reaction or a [3+2] cycloaddition reaction. It is preferred that the glucose is linked to an azide group and the detectable label is linked to a phosphine or cycloalkyne group. The detectable label is preferably a PET radionuclide label such as ¹⁸F.

### FIELD OF THE INVENTION

The invention relates to novel compounds, kits and method for use in medical imaging and therapy. The invention especially relates to alternatives for FDG, FLT and ¹¹C methionine.

### BACKGROUND TO THE INVENTION

The imaging modalities Positron Emission Tomography (PET) and Single Photon Emission Computed Tomography (SPECT) rely on radiolabelled pharmaceuticals to generate images.

PET records high-energy γ-rays emitted from within a subject. Positron emitting isotopes which are frequently used include ¹⁵O, ¹³ N, ¹¹C, and ¹⁸F, the latter is used as a substitute to hydrogen. Labelled molecular probes can be introduced into a subject and then PET imaging can follow the distribution and concentration of the injected molecules. SPECT imaging uses radiopharmaceuticals with an isotope that decays under gamma radiation emission. SPECT enables imaging of biological processes with kinetics in the order of hours to days. The most commonly used SPECT radionuclide is ^{99m}Tc.

In the area of molecular imaging, biological processes are imaged at a molecular level, which is achieved by the help of targeted imaging agents homing in on specific biological molecules in the body. Useful targets for both PET and SPECT imaging techniques are either highly-overexpressed molecules, which can be directly targeted by an imaging agent, or up-regulated pathways, which can be exploited to accumulate imaging agent in a diseased cell. Examples of such targets are the uptake pathways of the cell's building blocks. Building blocks are molecules that form the basis of molecules, structures and processes that are present in a cell. Examples of such building blocks are glucose, nucleo bases, amino acids and choline.

A current PET imaging technology using ¹⁸F labelled molecules for the diagnosis of cancer relies on the elevated accumulation of the simple sugar 2-fluoro-2-deoxy-glucose (FDG) in the tumour tissue relative to healthy tissue. Like glucose (one of the cell's building blocks), FDG is transported into cells by a glucose transporter and is rapidly converted into FDG-6-phosphate. However, as FDG lacks an hydroxyl group at the 2-position, it cannot undergo further phosphorylation and is trapped within the cell. Tumor cells have a higher glucose uptake than healthy cells and FDG accumulation is therefore also elevated, allowing the visualization of malignant lesions in a patient against a background uptake in normal tissue.

An important criterion for a successful imaging agent is that it exhibits a high target uptake while showing a rapid clearance (through renal and/or hepatobiliary systems) from non-target tissue and from the blood, so that a high contrast between the target and surrounding tissues can be obtained. This is in particular a challenge for nuclear probes, because these constantly produce signal by decaying. Consequently, a sufficient signal to background level has to be reached within several half-lives of the tracer.

One of the drawbacks of the use of FDG is the relatively short half-life, limiting its application to relatively fast processes. ¹⁸F has a half-life time of 110 minutes, so that the chemical reactions leading to the incorporation of the isotope into the parent molecule and subsequent introduction into a subject must take place relatively quickly. Furthermore, during the time it takes for FDG to accumulate in target cells and clear from non-target tissue, the isotope has already decayed to a considerable extent.

The fast pharmacokinetics of metabolism/proliferation imaging agents such as FDG generally matches their physical half-life. However, within the maximum timeframe of a few hours, these constructs can exhibit poor accumulation in slow growing tumors, small tumors, and tumors in dense tissue or with low blood flow. The accumulated signals in these tumors are often insufficient to be detectable over the background signal in non-target tissue and blood. Furthermore, accumulation in the clearance pathway, like hepatobiliary or kidney, can obscure the tissue of interest (e.g. in the case the bladder obscuring prostate cancer).

Therefore there is a need for alternatives to FDG, which allow the build up of the targeting molecule, such as glucose, before the radioactive compound is introduced. One of the aims of such alternatives is increasing the signal to noise ratio.

### SUMMARY OF THE INVENTION

It is an object of the invention to provide FDG alternatives and other imaging tools suitable for use in imaging and diagnostics. It has surprisingly been found that azide, phosphine or alkyne labelled analogues of traditional building blocks can easily be incorporated into metabolic pathways. These reactive groups can be covalently linked to a detectable label by reaction with a detectable label comprising the complementary group of the azide, phosphine or alkyne to react in a Staudinger ligation reaction or a [3+2] cycloaddition.

Therefore the invention in a first aspect relates to an imaging agent suitable for medical imaging techniques comprising a composition comprising a detectable label and at least one group selected from phosphine, alkyne and azide, preferably cycloalkyne or phosphine.

In further aspects the invention relates to a pharmaceutical composition comprising an azide moiety, a kit for medical imaging and a diagnostic method wherein the imaging agent is used.

### DETAILED DESCRIPTION OF THE INVENTION

The present invention will be described with respect to particular embodiments and with reference to certain drawings but the invention is not limited thereto but only by the claims. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated.

It is furthermore to be noticed that the term “comprising”, used in the description and in the claims, should not be interpreted as being restricted to the means listed thereafter; it does not exclude other elements or steps. Thus, the scope of the expression “a device comprising means A and B” should not be limited to devices consisting only of components A and B. It means that with respect to the present invention, the only relevant components of the device are A and B.

“Building blocks” are defined as molecules that are involved in pathways in a cell such as metabolic pathways. Building blocks may form part of molecules that are present in the cell such as sugars, DNA, RNA, peptides, lipids, proteins. Metabolic tracers and precursors are also referred to as building blocks. Examples of build blocks are glucose, nucleo bases, amino acids, fatty acids, acetates and choline.

Nucleo bases are the parts of RNA and DNA that are involved in pairing up. A nucleobase covalently bound to the 1′ carbon of a ribose or deoxyribose is called a nucleoside, and a nucleoside with one or more phosphate groups attached at the 5′ carbon is called a nucleotide. Examples of nucelobases are thymine, uracil, guanine, cytosine.

A “primary target” as used in the present invention relates to a target to be detected by imaging or a target for therapy. For example, a primary target can be any molecule, which is present in an organism, tissue or cell. Targets for imaging include cell surface targets, e.g. receptors, glycoproteins; structural proteins, e.g. amyloid plaques; intracellular targets, e.g. surfaces of Golgi bodies, surfaces of mitochondria, RNA, DNA, enzymes, components of cell signaling pathways; and/or foreign bodies, e.g. pathogens such as viruses, bacteria, fungi, yeast or parts thereof. Examples of primary targets include compounds such as proteins of which the presence or expression level is correlated with a certain tissue or cell type or of which the expression level is up regulated or downreguated in a certain disorder. According to a particular embodiment of the present invention, the primary target is a protein such as a receptor. Alternatively, the primary target may be a metabolic pathway, which is up regulated during a disease, e.g. infection or cancer, such as DNA synthesis, protein synthesis, membrane synthesis and saccharide uptake. In diseased tissues, above-mentioned markers can differ from healthy tissue and offer unique possibilities for early detection, specific diagnosis and therapy especially targeted therapy.

A “pre-targeting approach” in the context of the invention is a method wherein in a first step a composition that binds to a primary target is administered into a body or in vitro in a cellular system, in a seconds step a labeled composition that binds specifically to the composition that is bound to the primary target is administered.

The current invention provides an alternative to the well-known FDG detectable label for PET imaging.

In a first aspect the invention relates to an imaging agent suitable for medical imaging techniques comprising a composition comprising a detectable label and at least one group selected from phosphine, alkyne and azide, preferably cycloalkyne or phosphine. In this context, the phrase “composition comprising a detectable label and a group selected from . . . ” encompasses the embodiment where the detectable label and the group are linked by covalent or other interactions, either directly or indirectly, e.g. via a linker, and also the embodiment where these two components form part of the same non-separable system such as where the detectable label is incorporated in a shell, which shell is coated with at least one such group.

Without wishing to be bound by any theory, the invention is based on the known ligation reactions wherein azides take part. These are the Staudinger ligation and the [3+2] cycloaddition. The present invention provides a solution to the above mentioned limitations of current targeted imaging, using the [3+2] cycloaddition or Staudinger ligation which are covalent ligations, especially biocompatible covalent ligations. The Staudinger ligation and the [3+2] cycloaddition are selective chemical and bioorthogonal reactions.

The use of a biocompatible direct covalent reaction between two molecules, which does not occur in nature, solves the drawbacks encountered with recognition mechanisms based on non-covalent reactions in different applications. More particularly, it represents a number of advantages of particular interest in pre-targeting and represents a powerful new tool in molecular imaging.

With the methods of the present invention, two participating functional groups, e.g. azide and phosphine or alkyne, are used which equal the tremendous selectivity of non-covalent recognition events that occur in many biological processes, such as antibody-antigen binding. In accordance with an aspect of the present invention two participating functional groups are selected that have a finely tuned reactivity so that interference with coexisting functionality is avoided. In accordance with a further aspect of the present invention reactive partners are selected which are abiotic, form a stable adduct under physiological conditions, and recognize only each other while ignoring their cellular/physiological surroundings, i.e. they are bio-orthogonal. The demands on selectivity imposed by a biological environment preclude the use of most other conventional reactions.

Using the method and compounds of the present invention, imaging probes can be rapidly excreted from the body, due to their small size, e.g. through the kidneys, and can provide the desired high tumor accumulation with relatively low non-target accumulation. In nuclear medical imaging the concept of pre-targeting is advantageous. The pre-targeting step can be carried out as long as needed to achieve optimal target-uptake without using radioactive isotopes, while a second targeting step using a radioactive isotope, coupled to a small azide, phosphine or alkyne, can be carried out fast. This generally leads to an improved signal to noise ratio. Moreover, the present invention is particularly suitable for use in multimodal imaging, optionally using different imaging agents or different types of radionuclides to visualize the same target.

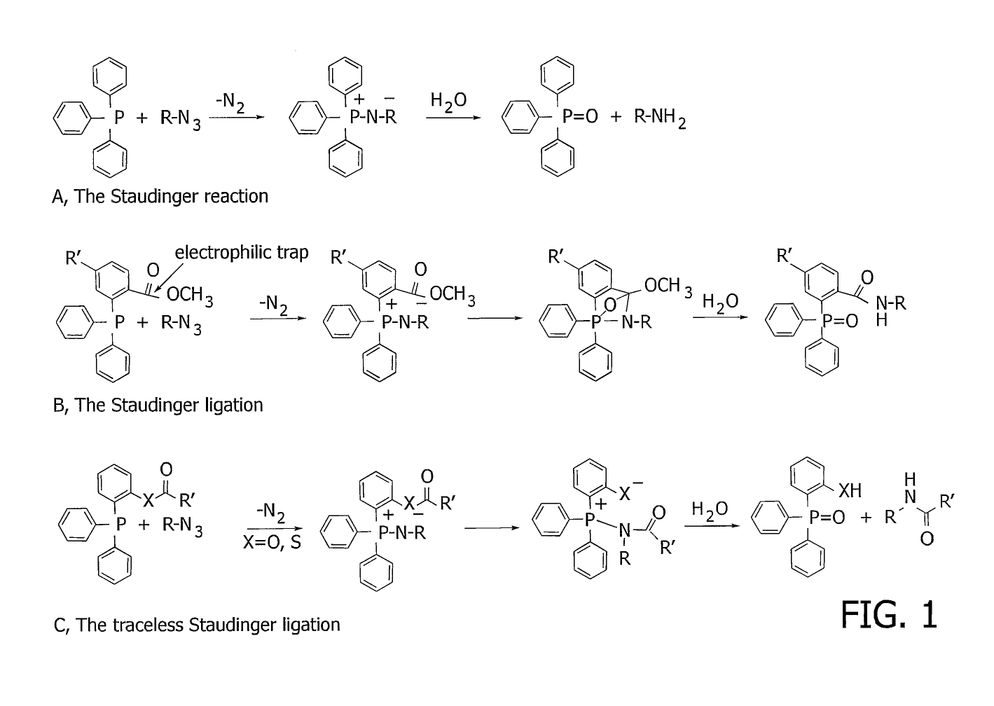

A chemoselective ligation, based on the classical Staudinger reaction between an azide and a phosphine (scheme A of FIG. 1), was applied by Bertozzi and co-workers to study cell surface glycosylation [reviewed in Kohn & Breinbauer (2004) Angew. Chem. Int. Ed. 43, 3106-3116].

A further modification is called the traceless Staudinger ligation and is depicted in scheme B of FIG. 1. Using the Staudinger ligation, Bertozzi and co-workers have demonstrated that N-azidoacetylmannosamine (ManNAz) was metabolically converted to the corresponding sialic acid and incorporated into cell surface glycoconjugates. The azide was available on the cell surface for Staudinger ligation with exogenous phosphine reagents. Control experiments revealed that neither azide reduction by endogenous monothiols (such as glutathione) nor the reduction of disulfides on the cell surface by the phosphine probe takes place.

In the [3+2] cycloaddition, an azide reacts with an alkyne, preferably a cycloalkyne to form a triazole adduct. Especially for cycloalkynes this reaction can take place without a catalyst such as a Cu catalyst, because of the strain present in the cycloalkyne ring.

Compounds of the invention are incorporated into a precursor molecule (also referred to as building blocks) to be incorporated into biomolecules or modified by the metabolism of the cell, trapping the molecules in or on the cell. In this way, general metabolic pathways can be targeted. The above-described phosphines, alkynes or azides are linked e.g. to sugars, amino acids, nucleo bases, fatty acids, choline or acetate, which can then be administered to the cell or organism and are incorporated into biomolecules and/or trapped in the cell by the normal metabolism. Examples of such incorporation into living organisms, eukaryotic cultivated cells or recombinant protein expression systems (bacteria, yeasts, higher eukaryotes), are described in the art [Lemieux et al 2003 cited above, Hang et al. (2003) Proc Natl. Acad Sci. USA 100, 14846-14851; Wang et al. (2003) Bioconjugate Chem. 14, 697-701].

In a particular embodiment of this aspect of the invention a metabolic pathway, which is upregulated during a disease, like infection/inflammation or cancer, is targeted. Components which can be upregulated in disease conditions include for example DNA, protein, membrane synthesis and sacharide uptake. Suitable building blocks to label these elements include azide-labeled amino acids, sugars, nucleobases and choline and acetate. These azide labeled building blocks are functionally analogous to the currently used metabolic tracers [¹¹C]-methionine, [¹⁸F]-fluorodeoxyglucose (FDG), deoxy-[¹⁸F]-fluorothymidine (FLT), [¹¹C]-acetate and [¹¹C]-choline. Cells with a high metabolism or proliferation have a higher uptake of these building blocks. Azide-derivatives can enter these pathways and accumulate in and/or on cells. After sufficient build-up and clearance of free building block, an imaging probe, e.g. a composition comprising a radioactive label and a (cell permeable) Staudinger phosphine or cycloalkyne, is sent in to bind the accumulated azide metabolite. The advantage over normal FDG-type imaging is that there is ample time to allow high build up of the targeting probe before radioactivity is allowed to bind, thus increasing the signal to noise ratio. Alternatively, a metabolic pathway and/or metabolite that is specific for a disease could be targeted.

In a preferred embodiment the imaging agent comprises a detectable label which comprises an alkyne or phosphine group to be partners in a reaction with azide, especially the [3+2] cycloaddition or Staudinger ligation respectively.

Optionally the imaging agent further comprises antioxidants, an aqueous medium, preferably a physiological salt solution, to enable easy administration.

The detectable label may be any suitable imaging label such as MRI-imageable agents, spin labels, optical labels, ultrasound-responsive agents, X-ray responsive agents, radionuclides, (bio) luminescent and FRET-type dyes. What is of high relevance to the invention is that the detectable label is directly suitable for imaging, without a further round of administration of a labeled antibody or the like being necessary. This is contrary to for example the known reactions where a phosphine probe comprising a Flag peptide, is administered to mice that previously were dosed with azide labeled peracetylated mannose (Nature volume 430, 2004, page 873-877). Imaging requires in this set up a further treatment with a fluorescein isothiocyanate-labelled anti-Flag antibody. This is a complicated multi-step process that does not provide the desired efficiency. Also the use of antibodies in the last step may not be desired because these may either lead to immunogenic reactions or may be too large to arrive at the desired cellular compartments.

Because of the relatively short half-times of most radionuclides, it was found that the invention is of special benefit for use in compounds where the detectable label is a radionuclide. Therefore in a preferred embodiment the detectable label is a radionuclide, preferably selected from the group comprising ³H, ¹¹C, ¹³N, ¹⁵O, ¹⁸F, ⁵¹Cr, ⁵²Fe, ^{52m}Mn, ⁵⁵Co, ⁶⁰Cu, ⁶¹Cu, ⁶²Zn, ⁶²Cu, ⁶³Zn, ⁶⁴Cu, ⁶⁶Ga, ⁶⁷Ga, ⁶⁸Ga, ⁷⁰As, ⁷¹As, ⁷²As, ⁷⁴As, ⁷⁵Se, ⁷⁵Br, ⁷⁶Br, ⁷⁷Br, ^{80m}Br, ^{82m}Br, ⁸²Rb, ⁸⁶Y, ⁸⁸Y, ⁸⁹Sr, ⁸⁹Zr, ⁹⁷Ru, ^{99m}Tc, ¹¹⁰In, ¹¹¹In, ^{113m}In, ^{114m}In, ^{117m}Sn, ¹²⁰I, ¹²²Xe, ¹²³I, ¹²⁴I, ¹²⁵I, ¹³¹I, ¹⁶⁶Ho, ¹⁶⁷Tm, ¹⁶⁹Yb, ^{193m}Pt, ^{195m}Pt, ²⁰¹Tl, ²⁰³Pb. More preferred the detectable label is selected from ¹⁸F, ¹²³I, ¹¹C. Most preferred the detectable label is ¹⁸F.

In one embodiment the detectable labels are small size organic PET and SPECT labels, such as ¹⁸F, ¹¹C or ¹²³I. Due to their small size, organic PET or SPECT labels, e.g. ¹⁸F, ¹¹C, or ¹²³I, are ideally suited for monitoring intracellular events as they do not greatly affect the properties of the targeting device in general and its membrane transport in particular. Likewise, the azide moiety is small and can be used as label for tracking cellular uptake and processing of building blocks of interest. An imaging probe comprising a PET label and triphenylphosphine is lipophilic and able to passively diffuse in and out of cells until it finds its binding partner. Moreover, both components do not preclude crossing of the blood brain barrier and thus allow imaging of regions in the brain.

Optionally the building block itself is already labeled with an imaging label. Preferably this label is different from the label that is introduced in a next step. Administration of the building block with label such as FDG functionalised with azide, gives rise to an FDG like image, which may in a second step be overlayed with the image that is obtained from a second targeting step with a labeled phosphine. This combination of two imaging labels, one being present in the building block and the other in the phosphine or alkyne that is administered thereafter, has as potential advantages better target localization, artifact elimination, delineation of non relevant clearance and other pharmacokinetic pathways.

Optionally the imaging agent comprises at least 2, preferably at least 3, more preferred from 2 to 5 detectable labels which may be the same or different, each comprising a group selected from phosphine, alkyne or azide. This enables multi modality imaging.

The invention further relates to a pharmaceutical composition comprising an azide, phosphine or cycloalkyne derivative of a building block selected from the group comprising sugars, amino acids, nucleo bases, fatty acids, choline or acetate or a combination thereof, and a pharmaceutically acceptable carrier. This azide, phosphine or alkyne moiety is a partner in the Staudinger ligation or [3+2] cycloaddtion with the detectable label compound present in the imaging agent. In a highly preferred embodiment, the derivative is an azide derivate. Such groups are small and were found to allow easy uptake in cellular metabolism.

Examples of pharmaceutically acceptable carriers include physiological salt solutions.

In a further aspect the invention relates to a method for preparing this pharmaceutical composition, which comprises mixing an azide derivative of a building block selected from the group comprising sugars, amino acids, nucleo bases, fatty acids, choline or acetate or a combination thereof with a pharmaceutically acceptable carrier.

In a most preferred embodiment, the building block is glucose and the derivative is an azide derivative.

In another aspect the invention relates to a kit for targeted medical imaging comprising:

at least one composition comprising a detectable label and at least one group selected from phosphine, cycloalkyne and azide;

a building block selected from the group comprising sugars, amino acids, nucleo bases, fatty acids, choline or acetate, the building block comprising a group selected from phosphine, alkyne and azide, which group is complementary to the group present in the composition comprising the detectable label, such that the detectable label and the building bock are partners in the Staudinger ligation or the [3+2] cycloaddition reaction via their respective reactive groups.

In this kit it is preferred that the building block comprises an azide group and the detectable label comprises a phosphine or alkyne group.

The building block is preferably selected from the group comprising sugar, amino acid and nucleo base. In a preferred embodiment, the building block is sugar, especially glucose. Glucose and analogues thereof accumulate in tumour tissue.

In a highly preferred embodiment of the invention, glucose comprising an azide group is incorporated into body tissue and accumulates in tumour tissue. Ligation of the azide group to a phosphine or alkyne moiety of a detectable label, will enable on imaging, the localisation of the tumour. The covalent bond that is a characteristic of the reaction product of a Staudinger ligation and a [3+2] cycloaddition in combination with the bioorthogonal nature of the reaction, makes these azide-based reactions highly beneficial for use in imaging.

Optionally, the kit of the invention further comprises a therapeutic agent. Optionally this agent is linked to the detectable label either permanently or in such a way that on reaction with the counterpart in the Staudinger ligation or [3+2] cycloaddition, the therapeutic agent is released. In this embodiment of the invention, a direct targeting of the therapeutic agent is possible.

Preferably the kit is accompanied by instructions for use in medical imaging comprising as a first step administration of the building block to a subject and as a second step administration of the detectable label and as a third step imaging.

The imaging agents and pharmaceutical compounds of the current invention are suitable for use in a diagnostic method or a method of treatment of a specific disease.

In a further aspect the invention relates to a diagnostic method using a detectable label and a building block, comprising the steps of:

a) administering to a subject or sample a building block selected from the group comprising sugar, amino acid and nucleobase, fatty acids, choline or acetate, the building block comprising a group selected from phosphine, alkyne and azide,

b) administering to the same subject or sample at least one composition comprising a detectable label comprising a group selected from phosphine, alkyne, preferably cycloalkyne, and azide, which group is complementary to the group present in the building block, such that the detectable label and the building block are partners in the Staudinger ligation or the [3+2] cycloaddition reaction;

c) imaging of the detectable label.

The invention is especially suitable for use as alternative to FDG aided imaging.

In this specific embodiment, a composition comprising glucose which comprises an azide group, is administered to a subject or tissue either in vivo or in vitro. Optionally an incubation period follows the administration to allow for incorporation of the azide containing glucose in the cellular system of the subject or tissue. In a next step, a phosphine or alkyne which is linked to detectable label ¹⁸F, is administered to the subject or tissue. A Staudinger ligation or a [3+2] cycloaddition will provide a covalent bond between the glucose and the label thereby enabling PET imaging of the glucose. In this embodiment, the imaging agent is a composition comprising ¹⁸F linked to a phosphine or alkyne and the glucose labelled with azide is a pharmaceutical composition. Preferably these two components are part of a kit suitable for medical imaging.

In the covalent reactions the following moieties take part.

The first is an azide moiety. Molecules comprising an azide and suitable for use in the present invention, as well as methods for producing azide-comprising molecules suitable for use in the present invention are known in the art.

Suitable alkynes are especially cycloalkynes for use in the present invention. Especially suitable cycloalkynes are those, which have sufficient ring strain to lead to a reaction with azide, which takes place without the need for a catalyst. Especially suitable cycloalkynes are those selected from the group comprising at least 6 carbon atoms. Cyclooctyne is the most preferred cycloalkyne for use in the current invention. Optionally the alkyne is substituted with electron withdrawing groups. This was found to increase the rate of the cycloaddition reaction with azides.

According to an embodiment of the present invention, the phosphine can be represented by the general structure:

Y—Z—PR₂R₃

wherein Z is an aryl group substituted with R₁, wherein R₁ is preferably in the ortho position on the aryl ring relative to the PR₂R₃; and wherein R₁ is an electrophilic group to trap, e.g., stabilize, an aza-ylide group, including, but not necessarily limited to, a carboxylic acid, an ester, e.g., an alkyl ester such as a lower alkyl ester, e.g. an alkyl having 1 to 4 carbon atoms, benzyl ester, aryl ester, substituted aryl ester, aldehyde, amide, e.g. an alkyl amide such as lower alkyl amide, e.g. an alkyl amide having 1 to 4 carbon atoms, aryl amide, an alkyl halide such as a lower alkyl halide, e.g. an alkyl halide having 1 to 4 carbon atoms, thioester, sulfonyl ester, an alkyl ketone such as a lower alkyl ketone e.g. an alkyl ketone having 1 to 4 carbon atoms, aryl ketone, substituted aryl ketone, halosulfonyl, nitrile, nitro and the like;

R₂ and R₃ are generally aryl groups, including substituted aryl groups, or alkyl groups, e.g., cyclohexyl groups where R₂ and R₃ may be the same or different, preferably the same; and

Y corresponds to one or a combination of a) a targeting moiety b) a detectable label, or c) a therapeutic compound. Y can be linked to the phosphine at a hydrogen or another reactive group at any position on the aryl group Z, and may also be linked to R2 or R3. e.g., para, meta, ortho; exemplary reactive groups include, but are not necessarily limited to, carboxyl, amine, e.g., alkyl amine such as a lower alkyl amine, e.g. comprising 1 to 4 carbon atoms, aryl amine, ester, e.g., alkyl ester such as a lower alkyl ester, e.g. comprising 1 to 4 carbon atoms, benzyl ester, aryl ester, substituted aryl ester, thioester, sulfonyl halide, alcohol, thiol, succinimidyl ester, isothiocyanate, iodoacetamide, maleimide, hydrazine, and the like. Alternatively, Y may be linked to the phosphine component through a linker.

The invention is illustrated by the following non-limiting examples.

### EXAMPLES

### Example 1

### Pre-Targeted Imaging of Tumors using Azide-Glucose

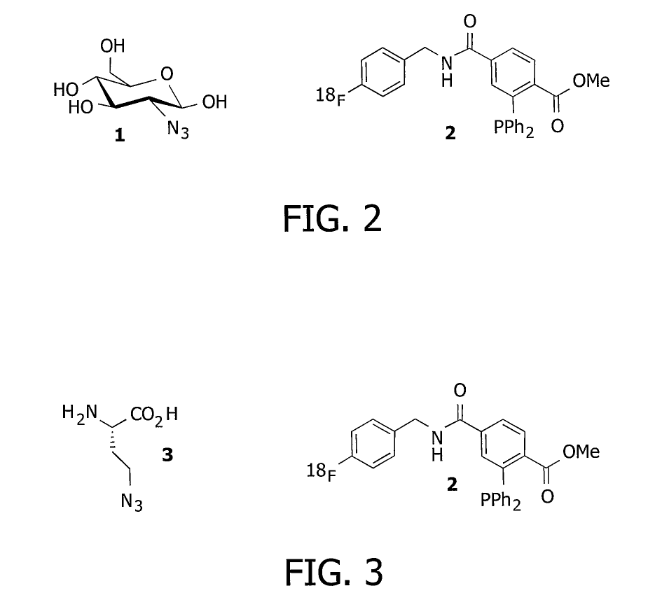

Reference is made to FIG. 2.

Azide-glucose probe 1 is administered systemically. After optimal accumulation in tissue with a high glucose uptake (e.g. tumor), and optimal clearance from non-target tissue and blood, ¹⁸F-labeled imaging probe 2 is administered. Construct 2 is conjugated to trapped 1 in cells via the Staudinger ligation. After clearance of non-bound 2 a PET image can be recorded, delineating the tumor location and activity.

### Example 2

### Pre-Targeted Imaging of Tumors using Azide-Amino Acids

Reference is made to FIG. 3. Azidohomoalanine (3) is recognized as a methionine surrogate by the translational apparatus of E. coli. [bertozzi_PNAS2002] and can serve as a metabolic/proliferation marker. Azidohomoalanine (3) is administered systemically. After optimal accumulation in tissue with a high amino acid uptake (e.g. tumor), and optimal clearance from non-target tissue and blood, ¹⁸F-labeled imaging probe 2 is administered. Construct 2 is conjugated to trapped 3 in cells via the Staudinger ligation. After clearance of non-bound 2 a PET image can be recorded, delineating the tumor location and activity.

### Example 3

### Pre-Targeted Imaging of Tumors using Azide-Nucleosides

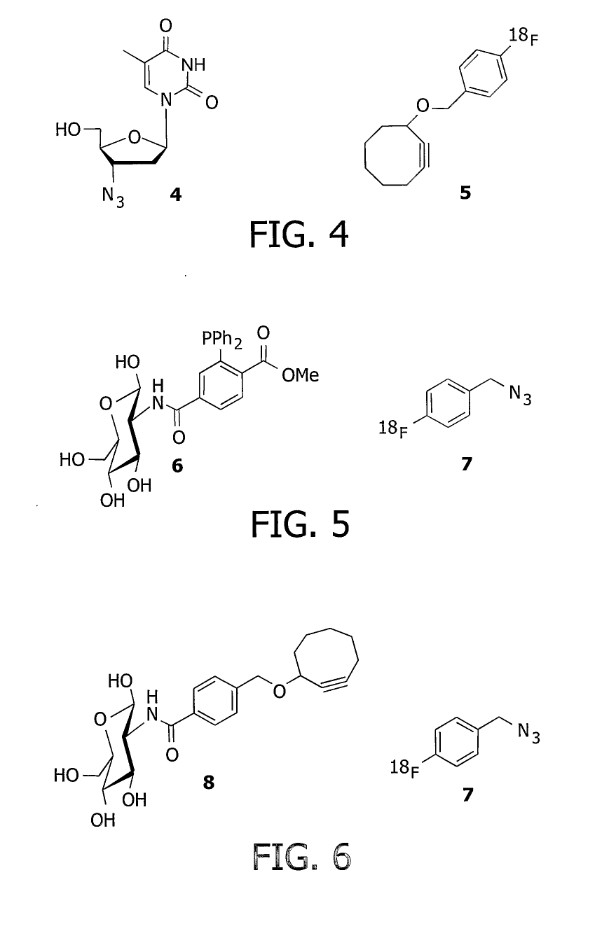

Reference is made to FIG. 4

Cell proliferation is increased in cancer, which leads to an increased DNA replication and therefore to an increased demand for nucleosides. Azide-modified thymidine 4 is administered and taken up by rapid-dividing cells. After optimal uptake in target cells, ¹⁸F-labelled cyclooctyne compound 5 is injected, which binds to trapped 4 via the [3+2] azide-alkyne cycloaddition. After clearance of non-bound 5 a PET image can be recorded, delineating the tumor location and activity.

### Example 4

### Pre-Targeted Imaging of Tumors using Glucose-Phosphine

Reference is made to FIG. 5.

Phosphine-glucose conjugate 6 can be recognized by the cellular glucose metabolism pathway. After systemic administration and optimal accumulation in tissue with a high glucose uptake (e.g. tumor), and optimal clearance from non-target tissue and blood, ¹⁸F-labeled imaging probe 7 is administered. Construct 7 is conjugated to trapped 6 in cells via the Staudinger ligation. After clearance of non-bound 7 a PET image can be recorded, delineating the tumor location and activity.

### Example 5

### Pre-Targeted Imaging of Tumors using Glucose-Cycloalkyne

Reference is made to FIG. 6

Cycloalkyne-glucose conjugate 8 can be recognized by the cellular glucose metabolism pathway. After systemic administration and optimal accumulation in tissue with a high glucose uptake (e.g. tumor), and optimal clearance from non-target tissue and blood, ¹⁸F-labeled imaging probe 7 is administered. Construct 7 is conjugated to trapped 8 in cells via [3+2] azide-alkyne cycloaddition. After clearance of non-bound 7 a PET image can be recorded, delineating the tumor location and activity.

1. Imaging agent suitable for medical imaging techniques comprising a composition comprising a detectable label and at least one group selected from phosphine, alkyne and azide.

2. Imaging agent according to claim 1 wherein the detectable label is a radionuclide label and the group is a phosphine or cycloalkyne moiety.

3. Use of a composition comprising a detectable label and a group selected from phosphine, alkyne and azide in the preparation of a diagnostic imaging composition.

4. Pharmaceutical composition comprising an azide, phosphine or alkyne derivative of a compound selected from the group of building blocks comprising sugars, amino acids, nucleo bases, fatty acids, choline or acetate or a combination thereof, and a pharmaceutically active carrier.

5. Pharmaceutical composition according to claim 4, comprising an azide derivative of glucose.

6. A method for preparing a composition according claim 4, which comprises mixing an azide derivative of a compound selected from the group of building blocks comprising sugars, amino acids, nucleo bases, fatty acids, choline or acetate or a combination thereof with a pharmaceutically acceptable carrier.

7. A kit for targeted medical imaging comprising:
at least one composition comprising a detectable label and at least one group selected from phosphine, alkyne and azide
a building block selected from the group comprising sugars, amino acids, nucleo bases, fatty acids, choline or acetate, the building block comprising a group selected from phosphine, alkyne and azide, which group is complementary to the group present in the composition comprising the detectable label, such that the detectable label and the building block are partners in the Staudinger ligation or the [3+2] cycloaddition reaction.

8. A kit according to claim 7 wherein the building block comprises an azide group and the detectable label comprises a phosphine or cycloalkyne group.

9. A kit according to aim 7 wherein the building block is glucose.

10. A kit according to claim 7 wherein the detectable label comprises ¹⁸F.

11. The kit according to claim 7, further comprising a therapeutic agent.

12. The kit according to claim 7 which further comprises instructions for use in medical imaging comprising as a first step administration of the building block to a subject and as a second step administration of the detectable label and as a third step imaging.

13. Diagnostic method using a detectable label and a building block, comprising the steps of:
a) administering to a subject or sample a building block selected from the group comprising sugar, amino acid, nucleobase, fatty acids, choline or acetate, the building block comprising a group selected from phosphine, alkyne and azide,
b) administering to the same subject or sample at least one composition comprising a detectable label comprising a group selected from phosphine, alkyne and azide, which group is complementary to the group present in the building block, such that the detectable label and the building block are partners in the Staudinger ligation or the [3+2] cycloaddition reaction
c) imaging of the detectable label.
